(19) United States
(12) Patent Application Publication
(10) Pub. No.: US 2010/0331387 A1
(43) Pub. Date: Dec. 30, 2010

(54) LYOPHILIZED PHARMACEUTICAL COMPOSITIONS

(75) Inventors: Thitiwan Buranachokpaisan, Bedminster, NJ (US); Wenlei Jiang, Boyds, MD (US); Wei-Qin Tong, Basking Ridge, NJ (US); Joseph Lawrence Zielinski, Florham Park, NJ (US); Jiahao Zhu, Whippany, NJ (US); Hans-Peter Zobel, Efringen-Kirchen (DE)

Correspondence Address: NOVARTIS;CORPORATE INTELLECTUAL PROPERTY, ONE HEALTH PLAZA 101/2, EAST HANOVER, NJ, 07936-1080, US

(73) Assignee: Novartis AG, Basel (CH)

(21) Appl. No.: 12/676,755

(22) PCT Filed: Sep. 18, 2008
(86) PCT No.: PCT/US08/76752

§ 371 Date: Sep. 15, 2010

Related U.S. Application Data
(60) Provisional application No. 60/973,830, filed on Sep. 20, 2007.

(51) Int. Cl. A61K 31/4045

(52) U.S. Cl. 514/415

(57) ABSTRACT

Pharmaceutical compositions that include a poorly water-soluble therapeutic compound, an aqueous solvent, an chelator/antioxidant, a buffer or buffer component, and a bulking agent. The pharmaceutical compositions can be orally ingested or administered parenterally. The pharmaceutical compositions can further be lyophilized to form a pharmaceutically acceptable cake that can be administered orally, e.g., as a solid oral dosage form; or reconstituted and administered parenterally, e.g. as a single i.v. bolus or iv infusion, or administered orally, e.g. as a drink solution.

### FIELD OF THE INVENTION

The present invention relates to lyophilized pharmaceutical compositions comprising hydroxamate compounds and the process of manufacture thereof.

### BACKGROUND OF THE INVENTION

Lyophilization, or more commonly known as freeze-drying, is a process which extracts water from a solution to form a granular solid or powder. The process is carried out by freezing the solution and subsequently extracting any water or moisture by sublimation under vacuum.

As compared to other drying techniques, lyophilization offers many advantages. For example, the quality of the substance being lyophilized is preserved while reducing the total weight of that substance. Furthermore, degradation of the therapeutic compound in a drug product is minimized since the lyophilized material is no longer exposed to water and air (especially when sealed in a vial that had been purged with a non-reactive gas such as nitrogen or argon); thus, the shelf life of the therapeutic compound is lengthened and enhanced. Additionally, lyophilized pharmaceutical compositions typically do not require particular conditions, such as refrigeration, for storage. Lyophilization is particularly useful for developing pharmaceutical drug products that are reconstituted and administered to a patient by injection, for example parenteral drug products. Alternatively, lyophilization is useful for developing oral drug products, especially fast melts or flash dissolve formulations.

Many new therapeutic compounds, including hydroxamate compounds, exhibit poor aqueous solubility and stability. To make such active pharmaceutical ingredients suitable for administration, e.g., parenterally, additional solubilizing excipients are often added. Often these poorly water-soluble therapeutic compounds are incorporated into systems that contain water and an organic solvent, called a cosolvent system. Although these liquid cosolvent systems increase solubility, they may do little to augment the stability of the therapeutic compound. As a result, lyophilization can be a preferred method to enhance both physical and chemical stability of the therapeutic compound.

### SUMMARY OF THE INVENTION

The present invention relates to a pharmaceutical composition comprising a hydroxamate compound; an chelator/antioxidant; a buffer or buffer component; and a bulking agent. In a particular embodiment of the present invention, the chelator/antioxidant comprises less than or equal to two percent weight/volume (w/v) of the composition. The buffer or the buffer component respectively comprises less than or equal to ten percent weight/volume (w/v) of the composition. Additionally, the bulking agent comprises one to fifty percent (w/v) of the composition.

In one aspect of the invention, a pharmaceutically acceptable cake resulting from the lyophilization of the pharmaceutical composition is described. In another aspect of the invention, the pharmaceutical composition is a pharmaceutically acceptable cake resulting from the lyophilization of the aforementioned solution. After this cake is reconstituted a solution is once again obtained; this solution is acceptable for parenteral administration, e.g., administered as an intravenous (i.v.) bolus dose; or oral administration, e.g., a drink. The pharmaceutically acceptable cake itself can be formed into a solid oral dosage form, e.g., a fast-melt or flash-dissolve tablet.

In a further aspect of the present invention, a process for making a pharmaceutically acceptable cake that can be reconstituted with water for parenteral administration is disclosed. This process comprises the steps of forming a solution comprising a hydroxamate compound; an chelator/antioxidant; a buffer; and a bulking agent; and lyophilizing the solution to form a pharmaceutically acceptable cake.

### DETAILED DESCRIPTION OF THE INVENTION

The present invention relates to a pharmaceutical composition that is suitable for parenteral or oral administration that comprises a therapeutic compound, i.e. hydroxamate compound; an chelator/antioxidant; a buffer; and a bulking agent. The present invention also relates to the pharmaceutically acceptable cake that results form the freeze-drying of the pharmaceutical composition. The pharmaceutically acceptable cake can be administered orally or parenterally after reconstitution, or swallowed orally without reconstitution. In addition to the aforementioned components, the solution can also optionally contain other excipients, such as pH adjusters, stabilizers, surfactants and other adjuvants recognized by one of ordinary skill in the art to be appropriate for such a composition. Examples of such excipients are described in Handbook of Pharmaceutical Excipients, 4^{th} Edition, Rowe et al., Eds., Pharmaceutical Press (2003).

As used herein, the term “pharmaceutical composition” means a solution containing a therapeutic compound to be administered to a mammal, e.g., a human. A pharmaceutical composition is “pharmaceutically acceptable” which refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

As used herein, the term “therapeutic compound” means a hydroxamate compound, and which is suitable for administration to a mammal, e.g., a human. Such therapeutic compounds should be administered in a “therapeutically effective amount”.

As used herein, the term “therapeutically effective amount” refers to an amount or concentration which is effective in reducing, eliminating, treating, preventing or controlling the symptoms of a disease or condition affecting a mammal. The term “controlling” is intended to refer to all processes wherein there may be a slowing, interrupting, arresting or stopping of the progression of the diseases and conditions affecting the mammal. However, “controlling” does not necessarily indicate a total elimination of all disease and condition symptoms, and is intended to include prophylactic treatment.

The appropriate therapeutically effective amount is known to one of ordinary skill in the art as the amount varies with the therapeutic compound being used and the indication which is being addressed. For example in accordance with the present invention, the therapeutic compound may be present in amount less than or equal to 10% (w/v).

The pharmaceutical composition or pharmaceutically acceptable cake, as described in detail below, will suitably contain between 0.1 mg and 100 mg of the therapeutic compound per unit dose, e.g., 0.1 mg, 1 mg, 5 mg, 10 mg, 20 mg, 25 mg, 50 mg or 100 mg per unit dose.

As used herein, the term “unit dose” means a single dose which is capable of being administered to a subject, and which can be readily handled and packaged, remaining as a physically and chemically stable unit dose comprising the therapeutic compound.

Therapeutic compounds that are particularly suited for the present invention are those that are poorly soluble in water. As used herein, the term “poorly water-soluble” refers to having a solubility in water at 20° C. of less than 1%, e.g., 0.01% (w/v), i.e., a “sparingly soluble to very slightly soluble drug” as described in Remington, The Science and Practice of Pharmacy, 19^{th} Edition, A. R. Gennaro, Ed., Mack Publishing Company, Vol. 1, p. 195 (1995).

Therapeutic compounds that are particularly suited for the present invention are pharmaceutical agents having the formula (I):

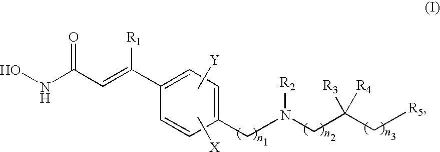

wherein
R₁ is H, halo, or a straight chain C₁-C₆alkyl (especially methyl, ethyl or n-propyl, which methyl, ethyl and n-propyl substituents are unsubstituted or substituted by one or more substituents described below for alkyl substituents);
R₂ is selected from H, C₁-C₁₀alkyl, (preferably C₁-C₆alkyl, e.g. methyl, ethyl or —CH₂CH₂—OH), C₄-C₉cycloalkyl, C₄-C₉heterocycloalkyl, C₄-C₉ heterocycloalkylalkyl, cycloalkylalkyl (e.g. cyclopropylmethyl), aryl, heteroaryl, arylalkyl (e.g. benzyl), heteroarylalkyl (e.g. pyridylmethyl), —(CH₂)ₙC(O) R₆, —(CH₂)ₙOC(O)R₆, amino acyl, HON—C(O)—CH═C(R₁)-aryl-alkyl- and —(CH₂)ₙR₇;
R₃ and R₄ are the same or different and independently H, C₁-C₆alkyl, acyl or acylamino, or
R₃ and R₄, together with the carbon to which they are bound, represent C═O, C═S or C═NR₈, or
R₂, together with the nitrogen to which it is bound, and R₃, together with the carbon to which it is bound, can form a C₄-C₉heterocycloalkyl, a heteroaryl, a polyheteroaryl, a non-aromatic polyheterocycle, or a mixed aryl and non-aryl polyheterocycle ring;
R₅ is selected from H, C₁-C₆alkyl, C₄-C₉cycloalkyl, C₄-C₉heterocycloalkyl, acyl, aryl, heteroaryl, arylalkyl (e.g., benzyl), heteroarylalkyl (e.g., pyridylmethyl), aromatic polycycles, non-aromatic polycycles, mixed aryl and non-aryl polycycles, polyheteroaryl, non-aromatic polyheterocycles, and mixed aryl and non-aryl polyheterocycles;
n, n₁, n₂ and n₃ are the same or different and independently selected from 0-6, when n₁ is 1-6, each carbon atom can be optionally and independently substituted with R₃ and/or R₄;
X and Y are the same or different and independently selected from H, halo, C₁-C₄alkyl, such as CH₃ and CF₃, NO₂, C(O)R₁, OR₉, SR₉, CN and NR₁₀R₁₁;
R₆ is selected from H, C₁-C₆alkyl, C₄-C₉cycloalkyl, C₄-C₉heterocycloalkyl, cycloalkylalkyl (e.g., cyclopropylmethyl), aryl, heteroaryl, arylalkyl (e.g., benzyl, 2-phenylethenyl), heteroarylalkyl (e.g., pyridylmethyl), OR₁₂ and NR₁₃R₁₄;
R₇ is selected from OR₁₅, SR₁₅, S(O)R₁₆, SO₂R₁₇, NR₁₃R₁₄, and NR₁₂SO₂R₆;
R₈ is selected from H, OR₁₅, NR₁₃R₁₄, C₁-C₆alkyl, C₄-C₉cycloalkyl, C₄-C₉heterocycloalkyl, aryl, heteroaryl, arylalkyl (e.g., benzyl), and heteroarylalkyl (e.g., pyridylmethyl);
R₉ is selected from C₁-C₄alkyl, e.g., CH₃ and CF₃, C(O)-alkyl, e.g., C(O)CH₃ and C(O)CF₃;
R₁₀ and R₁₁ are the same or different and independently selected from H, C₁-C₄alkyl, and —C(O)-alkyl;
R₁₂ is selected from H, C₁-C₆alkyl, C₄-C₉cycloalkyl, C₄-C₉heterocycloalkyl, C₄-C₉ heterocycloalkylalkyl, aryl, mixed aryl and non-aryl polycycle, heteroaryl, arylalkyl (e.g., benzyl), and heteroarylalkyl (e.g., pyridylmethyl);
R₁₃ and R₁₄ are the same or different and independently selected from H, C₁-C₆alkyl, C₄-C₉cycloalkyl, C₄-C₉heterocycloalkyl, aryl, heteroaryl, arylalkyl (e.g., benzyl), heteroarylalkyl (e.g., pyridylmethyl), amino acyl, or
R₁₃ and R₁₄, together with the nitrogen to which they are bound, are C₄-C₉heterocycloalkyl, heteroaryl, polyheteroaryl, non-aromatic polyheterocycle or mixed aryl and non-aryl polyheterocycle;
R₁₅ is selected from H, C₁-C₆alkyl, C₄-C₉ cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉ heterocycloalkyl, aryl, heteroaryl, polyheteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, aromatic polycycles, heteroaryl, arylalkyl, heteroarylalkyl, polyheteroaryl and NR₁₃R₁₄;
m is an integer selected from 0-6; and
Z is selected from O, NR₁₃, S and S(O),
   or a pharmaceutically acceptable salt thereof.

As appropriate, unsubstituted means that there is no substituent or that the only substituents are hydrogen.

Halo substituents are selected from fluoro, chloro, bromo and iodo, preferably fluoro or chloro.

Alkyl substituents include straight and branched C₁-C₆alkyl, unless otherwise noted. Examples of suitable straight and branched C₁-C₆alkyl substituents include methyl, ethyl, n-propyl, 2-propyl, n-butyl, sec-butyl, t-butyl and the like. Unless otherwise noted, the alkyl substituents include both unsubstituted alkyl groups and alkyl groups that are substituted by one or more suitable substituents, including unsaturation (i.e., there are one or more double or triple C—C bonds), acyl, cycloalkyl, halo, oxyalkyl, alkylamino, aminoalkyl, acylamino and OR₁₅, for example, alkoxy. Preferred substituents for alkyl groups include halo, hydroxy, alkoxy, oxyalkyl, alkylamino and aminoalkyl.

Cycloalkyl substituents include C₃-C₉cycloalkyl groups, such as cyclopropyl, cyclobutyl, cyclopentyl, cyclohexyl and the like, unless otherwise specified. Unless otherwise noted, cycloalkyl substituents include both unsubstituted cycloalkyl groups and cycloalkyl groups that are substituted by one or more suitable substituents, including C₁-C₆alkyl, halo, hydroxy, aminoalkyl, oxyalkyl, alkylamino, and OR₁₅, such as alkoxy. Preferred substituents for cycloalkyl groups include halo, hydroxy, alkoxy, oxyalkyl, alkylamino and aminoalkyl.

The above discussion of alkyl and cycloalkyl substituents also applies to the alkyl portions of other substituents, such as without limitation, alkoxy, alkyl amines, alkyl ketones, arylalkyl, heteroarylalkyl, alkylsulfonyl and alkyl ester substituents and the like.

Heterocycloalkyl substituents include 3- to 9-membered aliphatic rings, such as 4- to 7-membered aliphatic rings, containing from one to three heteroatoms selected from nitrogen, sulfur and oxygen. Examples of suitable heterocycloalkyl substituents include pyrrolidyl, tetrahydrofuryl, tetrahydrothiofuranyl, piperidyl, piperazyl, tetrahydropyranyl, morphilino, 1,3-diazapane, 1,4-diazapane, 1,4-oxazepane and 1,4-oxathiapane. Unless otherwise noted, the rings are unsubstituted or substituted on the carbon atoms by one or more suitable substituents, including C₁-C₆alkyl, C₄-C₉cycloalkyl, aryl, heteroaryl, arylalkyl (e.g., benzyl), and heteroarylalkyl (e.g., pyridylmethyl), halo, amino, alkyl amino and OR₁₅ , e.g., alkoxy. Unless otherwise noted, nitrogen heteroatoms are unsubstituted or substituted by H, C₁-C₄alkyl, arylalkyl (e.g., benzyl), and heteroarylalkyl (e.g., pyridylmethyl), acyl, aminoacyl, alkylsulfonyl and arylsulfonyl.

Cycloalkylalkyl substituents include compounds of the formula —(CH₂)ₙ₅-cycloalkyl wherein n5 is a number from 1-6. Suitable cycloalkylalkyl substituents include cyclopentylmethyl-, cyclopentylethyl, cyclohexylmethyl and the like. Such substituents are unsubstituted or substituted in the alkyl portion or in the cycloalkyl portion by a suitable substituent, including those listed above for alkyl and cycloalkyl.

Aryl substituents include unsubstituted phenyl and phenyl substituted by one or more suitable substituents, including C₁-C₆alkyl, cycloalkylalkyl (e.g., cyclopropylmethyl), O(CO)alkyl, oxyalkyl, halo, nitro, amino, alkylamino, aminoalkyl, alkyl ketones, nitrile, carboxyalkyl, alkylsulfonyl, aminosulfonyl, arylsulfonyl, and OR₁₅, such as alkoxy. Preferred substituents include including C₁-C₆alkyl, cycloalkyl (e.g., cyclopropylmethyl), alkoxy, oxyalkyl, halo, nitro, amino, alkylamino, aminoalkyl, alkyl ketones, nitrile, carboxyalkyl, alkylsulfonyl, arylsulfonyl, and aminosulfonyl. Examples of suitable aryl groups include C₁-C₄alkylphenyl, C₁-C₄alkoxyphenyl, trifluoromethylphenyl, methoxyphenyl, hydroxyethylphenyl, dimethylaminophenyl, aminopropylphenyl, carbethoxyphenyl, methanesulfonylphenyl and tolylsulfonylphenyl.

Aromatic polycycles include naphthyl, and naphthyl substituted by one or more suitable substituents, including C₁-C₆ alkyl, cycloalkylalkyl (e.g., cyclopropylmethyl), oxyalkyl, halo, nitro, amino, alkylamino, aminoalkyl, alkyl ketones, nitrile, carboxyalkyl, alkylsulfonyl, arylsulfonyl, aminosulfonyl and OR₁₅, such as alkoxy.

Heteroaryl substituents include compounds with a 5- to 7-membered aromatic ring containing one or more heteroatoms, e.g., from 1 to 4 heteroatoms, selected from N, O and S. Typical heteroaryl substituents include furyl, thienyl, pyrrole, pyrazole, triazole, thiazole, oxazole, pyridine, pyrimidine, isoxazolyl, pyrazine and the like. Unless otherwise noted, heteroaryl substituents are unsubstituted or substituted on a carbon atom by one or more suitable substituents, including alkyl, the alkyl substituents identified above, and another heteroaryl substituent. Nitrogen atoms are unsubstituted or substituted, e.g., by R₁₃; especially useful N substituents include H, C₁-C₄acyl, aminoacyl and sulfonyl.

Arylalkyl substituents include groups of the formula —(CH₂)ₙ₅-aryl, —(CH₂)ₙ₅₋₁—(CHaryl)-(CH₂)ₙ₅-aryl or —(CH₂)ₙ₅₋₁CH(aryl)(aryl), wherein aryl and n5 are as defined above. Such arylalkyl substituents include benzyl, 2-phenylethyl, 1-phenylethyl, tolyl-3-propyl, 2-phenylpropyl, diphenylmethyl, 2-diphenylethyl, 5,5-dimethyl-3-phenylpentyl and the like. Arylalkyl substituents are unsubstituted or substituted in the alkyl moiety or the aryl moiety or both as described above for alkyl and aryl substituents.

Heteroarylalkyl substituents include groups of the formula —(CH₂)ₙ₅-heteroaryl, wherein heteroaryl and n5 are as defined above and the bridging group is linked to a carbon or a nitrogen of the heteroaryl portion, such as 2-, 3- or 4-pyridylmethyl, imidazolylmethyl, quinolylethyl, and pyrrolylbutyl. Heteroaryl substituents are unsubstituted or substituted as discussed above for heteroaryl and alkyl substituents.

Amino acyl substituents include groups of the formula —C(O)—(CH₂)ₙ—C(H)(NR₁₃R₁₄)—(CH₂)ₙ—R₅, wherein n, R₁₃, R₁₄ and R₅ are described above. Suitable aminoacyl substituents include natural and non-natural amino acids such as glycinyl, D-tryptophanyl, L-lysinyl, D- or L-homoserinyl, 4-aminobutryic acyl, ±-3-amin-4-hexenoyl.

Non-aromatic polycycle substituents include bicyclic and tricyclic fused ring systems where each ring can be 4-9 membered and each ring can contain 0, 1 or more double and/or triple bonds. Suitable examples of non-aromatic polycycles include decalin, octahydroindene, perhydrobenzocyclohepterie, perhydrobenzo-[f]-azulene. Such substituents are unsubstituted or substituted as described above for cycloalkyl groups.

Mixed aryl and non-aryl polycycle substituents include bicyclic and tricyclic fused ring systems where each ring can be 4- to 9-membered and at least one ring is aromatic. Suitable examples of mixed aryl and non-aryl polycycles include methylenedioxyphenyl, bis-methylenedioxyphenyl, 1,2,3,4-tetrahydronaphthalene, dibenzosuberane, dihdydroanthracene, 9H-fluorene. Such substituents are unsubstituted or substituted by nitro or as described above for cycloalkyl groups.

Polyheteroaryl substituents include bicyclic and tricyclic fused ring systems where each ring can independently be 5- or 6-membered and contain one or more heteroatom, e.g., 1, 2, 3 or 4 heteroatoms, chosen from O, N or S such that the fused ring system is aromatic. Suitable examples of polyheteroaryl ring systems include quinoline, isoquinoline, pyridopyrazine, pyrrolopyridine, furopyridine, indole, benzofuran, benzothiofuran, benzindole, benzoxazole, pyrroloquinoline and the like. Unless otherwise noted, polyheteroaryl substituents are unsubstituted or substituted on a carbon atom by one or more suitable substituents, including alkyl, the alkyl substituents identified above and a substituent of the formula —O—(CH₂CH═CH(CH₃)(C₂))₁₋₃H. Nitrogen atoms are unsubstituted or substituted, e.g., by R₁₃; especially useful N substituents include H, C₁-C₄alkyl, acyl, aminoacyl and sulfonyl.

Non-aromatic polyheterocyclic substituents include bicyclic and tricyclic fused ring systems where each ring can be 4- to 9-membered, contain one or more heteroatom, e.g., 1, 2, 3 or 4 heteroatoms, chosen from O, N or S and contain zero or one or more C—C double or triple bonds. Suitable examples of non-aromatic polyheterocycles include hexitol, cis-perhydro-cyclohepta[b]pyridinyl, decahydro-benzo[f][1,4]oxazepinyl, 2,8-dioxabicyclo[3.3.0]octane, hexahydro-thieno[3,2-b]thiophene, perhydropyrrolo[3,2-b]pyrrole, perhydronaphthyridine, perhydro-1H-dicyclopenta[b,e]pyran. Unless otherwise noted, non-aromatic polyheterocyclic substituents are unsubstituted or substituted on a carbon atom by one or more substituents, including alkyl and the alkyl substituents identified above. Nitrogen atoms are unsubstituted or substituted, e.g., by R₁₃; especially useful N substituents include H, C₁-C₄alkyl, acyl, aminoacyl and sulfonyl.

Mixed aryl and non-aryl polyheterocycles substituents include bicyclic and tricyclic fused ring systems where each ring can be 4- to 9-membered, contain one or more heteroatom chosen from O, N or S, and at least one of the rings must be aromatic. Suitable examples of mixed aryl and non-aryl polyheterocycles include 2,3-dihydroindole, 1,2,3,4-tetrahydroquinoline, 5,11-dihydro-10H-dibenz[b,e][1,4]diazepine, 5H-dibenzo[b,e][1,4]diazepine, 1,2-dihydropyrrolo[3,4-b][1,5]benzodiazepine, 1,5-dihydro-pyrido[2,3-b][1,4]diazepin-4-one, 1,2,3,4,6,11-hexahydro-benzo[b]pyrido[2,3-e][1,4]diazepin-5-one. Unless otherwise noted, mixed aryl and non-aryl polyheterocyclic substituents are unsubstituted or substituted on a carbon atom by one or more suitable substituents, including, —N—OH, ═N—OH, alkyl and the alkyl substituents identified above. Nitrogen atoms are unsubstituted or substituted, e.g., by R₁₃; especially useful N substituents include H, C₁-C₄alkyl, acyl, aminoacyl and sulfonyl.

Amino substituents include primary, secondary and tertiary amines and in salt form, quaternary amines. Examples of amino substituents include mono- and di-alkylamino, mono- and di-aryl amino, mono- and di-arylalkyl amino, aryl-arylalkylamino, arkyl-arylamino, alkyl-arylalkylamino and the like.

Sulfonyl substituents include alkylsulfonyl and arylsulfonyl, e.g., methane sulfonyl, benzene, sulfonyl, tosyl and the like.

Acyl substituents include groups of the formula —C(O)—W, —OC(O)—W, —C(O)—O—W and —C(O)NR₁₃R₁₄, where W is R₁₆, H or cycloalkylalkyl.

Acylamino substituents include groups of the formula —N(R₁₂)C(O)—W, —N(R₁₂)C(O)—O—W and —N(R₁₂)C(O)—NHOH and R₁₂ and W are as defined above.

The R₂ substituent HON—C(O)—CH═O(R₁)-aryl-alkyl- is a group of the formula:

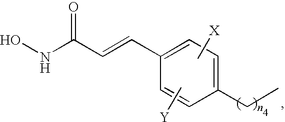

wherein
n₄ is 0-3; and
X and Y are as defined above.

Preferences for each of the substituents include the following;
R₁ is H, halo, or a straight chain C₁-C₄alkyl;
R₂ is selected from H, C₁-C₆ alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₃ and R₄ are the same or different and independently selected from H, and C₁-C₆alkyl, or
R₃ and R₄ together with the carbon to which they are bound, represent C═O, O═S or C═NR₈;
R₅ is selected from H, C₁-C₆alkyl, C₄-C₉cycloalkyl, C₄-C₉heterocycloalkyl, aryl, heteroaryl, arylalkyl, heteroarylalkyl, an aromatic polycycle, a non-aromatic polycycle, a mixed aryl and non-aryl polycycle, polyheteroaryl, a non-aromatic polyheterocycle, and a mixed aryl and non-aryl polyheterocycle;
n, n₁, n₂ and n₃ are the same or different and independently selected from 0-6, when n₁ is 1-6, each carbon atom is unsubstituted or independently substituted with R₃ and/or R₄;
X and Y are the same or different and independently selected from H, halo, C₁-C₄alkyl, CF₃, NO₂, C(O)R₁, OR₉, SR₉, CN and NR₁₀R₁₁;
R₆ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, OR₁₂ and NR₁₃R₁₄;
R₇ is selected from OR₁₅, SR₁₅, S(O)R₁₆, SO₂R₁₇, NR₁₃R₁₄ and NR₁₂SO₂R₆;
R₈ is selected from H, OR₁₅, NR₁₃R₁₄, C₁-C₆alkyl, C₄-C₉cycloalkyl, C₄-C₉heterocycloalkyl, aryl, heteroaryl, arylalkyl and heteroarylalkyl;
R₉ is selected from C₁-C₄alkyl and C(O)-alkyl;
R₁₀ and R₁₁ are the same or different and independently selected from H, C₁-C₄alkyl and —C(O)-alkyl;
R₁₂ is selected from H, C₁-C₆alkyl, C₄-C₉cycloalkyl, C₄-C₉heterocycloalkyl, aryl, heteroaryl, arylalkyl and heteroarylalkyl;
R₁₃ and R₁₄ are the same or different and independently selected from H, C₁-C₆alkyl, C₄-C₉cycloalkyl, C₄-C₉heterocycloalkyl, aryl, heteroaryl, arylalkyl, heteroarylalkyl and amino acyl;
R₁₅ is selected from H, C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₆ is selected from C₁-C₆alkyl, C₄-C₉cycloalkyl, C₄-C₉heterocycloalkyl, aryl, heteroaryl, arylalkyl, heteroarylalkyl and (CH₂)ₘZR₁₂;
R₁₇ is selected from C₁-C₆alkyl, C₄-C₉cycloalkyl, C₄-C₉heterocycloalkyl, aryl, heteroaryl, arylalkyl, heteroarylalkyl and NR₁₃R₁₄;
m is an integer selected from 0 to 6; and
Z is selected from O, NR₁₃, S, S(O).

Useful compounds of the formula (I) include those wherein each of R₁, X, Y, R₃, and R₄ is H, including those wherein one of n₂ and n₃ is 0 and the other is 1, especially those wherein R₂ is H or —CH₂—CH₂—OH.

One suitable genus of hydroxamate compounds are those of formula (Ia);

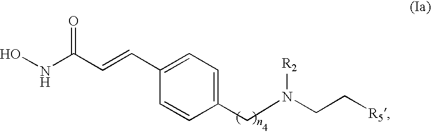

wherein
n₄ is 0-3;
R₂ is selected from H, C₁-C₆alkyl C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is heteroaryl, heteroarylalkyl (e.g., pyridylmethyl), aromatic polycycles, non-aromatic polycycles, mixed aryl and non-aryl polycycles, polyheteroaryl, or mixed aryl and non-aryl polyheterocycles,
   or a pharmaceutically acceptable salt thereof.

Another suitable genus of hydroxamate compounds are those of formula (Ia):

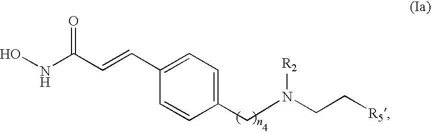

wherein
n₄ is 0-3;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
R₅′ is aryl, arylalkyl, aromatic polycycles, non-aromatic polycycles, and mixed aryl and non-aryl polycycles; especially aryl, such as p-fluorophenyl, p-chlorophenyl, p-O—C₁-C₄alkylphenyl, such as p-methoxyphenyl, and p-C₁-C₄alkylphenyl; and arylalkyl, such as benzyl, ortho, meta or para-fluorobenzyl, ortho, meta or para-chlorobenzyl, ortho, meta or para-mono, di- or tri-O—C₁-C₄alkylbenzyl, such as ortho, meta or para-methoxybenzyl, m,p-diethoxybenzyl, o,m,p-triimethoxybenzyl, and ortho, meta or para-mono, di- or tri-C₁-C₄alkylphenyl, such as p-methyl, m,m-diethylphenyl,
   or a pharmaceutically acceptable salt thereof.

Another interesting genus are the compounds of formula (Ib):

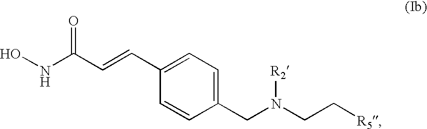

wherein
R₂ ′ is selected from H, C₁-C₆alkyl C₄-C₆cycloalkyl, cycloalkylalkyl (e.g., cyclopropylmethyl), —(CH₂)₂₋₄OR₂₁ where R₂₁ is H, methyl, ethyl, propyl, and i-propyl, and
R₅″ is unsubstituted 1H-indol-3-yl, benzofuran-3-yl or quinolin-3-yl, or substituted 1H-indol-3-yl, such as 5-fluoro-1H-indol-3-yl or 5-methoxy-1H-indol-3-yl, benzofuran-3-yl or quinolin-3-yl,
   or a pharmaceutically acceptable salt thereof.

Another interesting genus of hydroxamate compounds are the compounds of formula (Ic)

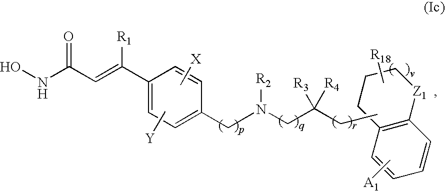

wherein
the ring containing Z₁ is aromatic or non-aromatic, which non-aromatic rings are saturated or unsaturated;
Z₁ is O, S or N—R₂₀;
R₁₈ is H, halo, C₁-C₆alkyl(methyl, ethyl, t-butyl), C₃-C₇cycloalkyl, aryl, e.g., unsubstituted phenyl or phenyl substituted by 4-OCH₃ or 4-CF₃, or heteroaryl, such as 2-furanyl, 2-thiophenyl or 2-, 3- or 4-pyridyl;
R₂₀ is H, C₁-C₆alkyl, C₁-C₆alkyl-C₃-C₉cycloalkyl (e.g., cyclopropylmethyl), aryl, heteroaryl, arylalkyl (e.g., benzyl), heteroarylalkyl (e.g., pyridylmethyl), acyl(acetyl, propionyl, benzoyl) or sulfonyl(methanesulfonyl, ethanesulfonyl, benzenesulfonyl, toluenesulfonyl);
A₁ is 1, 2 or 3 substituents which are independently H, C₁-C₆alkyl, —OR₁₉, halo, alkylamino, aminoalkyl, halo or heteroarylalkyl (e.g., pyridylmethyl),
R₁₉ is selected from H, C₁-C₆alkyl, C₄-C₉cycloalkyl, C₄-C₉heterocycloalkyl, aryl, heteroaryl, arylalkyl (e.g., benzyl), heteroarylalkyl (e.g., pyridylmethyl) and —(CH₂CH═CH(CH₃)(CH₂))₁₋₃H;
R₂ is selected from H, C₁-C₆alkyl, C₄-C₉cycloalkyl, C₄-C₉heterocycloalkyl, cycloalkylalkyl, aryl, heteroaryl, arylalkyl, heteroarylalkyl, —(CH₂)ₙC(O)R₆, amino acyl and —(CH₂)ₙR₇;
v is 0, 1 or 2;
p is 0-3; and
q is 1-5 and r is 0, or
q is 0 and r is 1-5,
   or a pharmaceutically acceptable salt thereof. The other variable substituents are as defined above.

Especially useful compounds of formula (Ic) are those wherein R₂ is H, or —(CH₂)ₚCH₂OH, wherein p is 1-3, especially those wherein R₁ is H, such as those wherein R₁ is H and X and Y are each H, and wherein q is 1-3 and r is 0 or wherein q is 0 and r is 1-3, especially those wherein Z₁ is N—R₂₀. Among these compounds R₂ is preferably H or —CH₂—CH₂—OH and the sum of q and r is preferably 1.

Another interesting genus of hydroxamate compounds are the compounds of formula (Id)

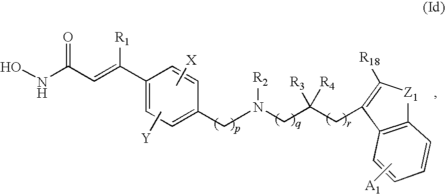

wherein
Z is O, S or N—R₂₀;
R₁₈ is H, halo, C₁-C₆alkyl(methyl, ethyl, t-butyl), C₃-C₇cycloalkyl, aryl, for example, unsubstituted phenyl or phenyl substituted by 4-OCH₃ or 4-CF₃ or heteroaryl;
R₂₀ is H, C₁-C₆alkyl, C₁-C₆alkyl-C₃-C₉cycloalkyl (e.g., cyclopropylmethyl), aryl, heteroaryl, arylalkyl (e.g., benzyl), heteroarylalkyl (e.g., pyridylmethyl), acyl(acetyl, propionyl, benzoyl) or sulfonyl(methanesulfonyl, ethanesulfonyl, benzenesulfonyl, toluenesulfonyl);
A₁ is 1, 2 or 3 substituents which are independently H, C₁-C₆, —OR₁₉ or halo,
R₁₉ is selected from H, C₁-C₆alkyl, C₄-C₉cycloalkyl, C₄-C₉heterocycloalkyl, aryl, heteroaryl, arylalkyl (e.g., benzyl), and heteroarylalkyl (e.g., pyridylmethyl);
p is 0-3; and
q is 1-5 and r is 0, or
q is 0 and r is 1-5,
   or a pharmaceutically acceptable salt thereof. The other variable substituents are as defined above.

Especially useful compounds of formula (Id) are those wherein R₂ is H, or —(CH₂)ₚCH₂OH, wherein p is 1-3, especially those wherein R₁ is H, such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

The present invention further relates to compounds of the formula (Ie):

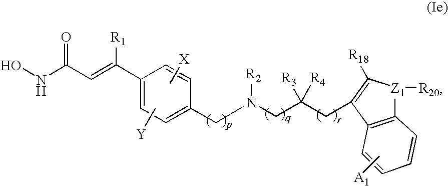

or a pharmaceutically acceptable salt thereof. The variable substituents are as defined above.

Especially useful compounds of formula (Ie) are those wherein R₁₈ is H, fluoro, chloro, bromo, a C₁-C₄alkyl group, a substituted C₁-C₄alkyl group, a C₃-C₇cycloalkyl group, unsubstituted phenyl, phenyl substituted in the para position, or a heteroaryl (e.g., pyridyl) ring.

Another group of useful compounds of formula (Ie) are those wherein R₂ is H, or —(CH₂)ₚCH₂OH, wherein p is 1-3, especially those wherein R₁ is H, such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Another group of useful compounds of formula (Ie) are those wherein R₁₈ is H, methyl, ethyl, t-butyl, trifluoromethyl, cyclohexyl, phenyl, 4-methoxyphenyl, 4-trifluoromethylphenyl, 2-furanyl, 2-thiophenyl, or 2-, 3- or 4-pyridyl wherein the 2-furanyl, 2-thiophenyl and 2-, 3- or 4-pyridyl substituents are unsubstituted or substituted as described above for heteroaryl rings; R₂ is H, or —(CH₂)ₚCH₂OH, wherein p is 1-3; especially those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1.

Those compounds of formula (Ie), wherein R₂₀ is H or C₁-C₆alkyl, especially H, are important members of each of the subgenuses of compounds of formula (Ie) described above.

N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(1H-indol-3-yl)ethyl]-amino]methyl]phenyl]-2E-2-propenamide and N-hydroxy-3-[4-[[[2-(2-methyl-1H-indol-3-yl)-ethyl]-amino]methyl]phenyl]-2E-2-propenamide, or a pharmaceutically acceptable salt thereof, are important compounds of formula (Ie).

The present invention further relates to the compounds of the formula (If):

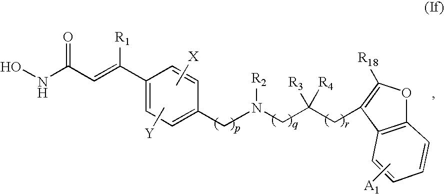

or a pharmaceutically acceptable salt thereof. The variable substituents are as defined above.

Useful compounds of formula If are those wherein R₂ is H, or —(C₁)ₚCH₂OH, wherein p is 1-3, especially those wherein R₁ is H; such as those wherein R₁ is H and X and Y are each H, and wherein q is 1-3 and r is 0 or wherein q is 0 and r is 1-3. Among these compounds R₂ is preferably H or —CH₂—CH₂—OH and the sum of q and r is preferably 1

N-hydroxy-3-[4-[[[2-(benzofur-3-yl)-ethyl]-amino]methyl]phenyl]-2E-2-propenamide, or a pharmaceutically acceptable salt thereof.

The compounds described above are often used in the form of a pharmaceutically acceptable salt. Pharmaceutically acceptable salts include, when appropriate, pharmaceutically acceptable base addition salts and acid addition salts, e.g., metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylate and benzene sulfonic acid salts.

A preferred therapeutic compound of the present invention is N-hydroxy-3-[4-[[[2-(2-methyl-1H-indol-3-yl)-ethyl]-amino]methyl]phenyl]-2E-2-propenamide, or a pharmaceutically acceptable salt thereof, preferably the lactate salt.

Non-limiting examples of buffers used in the present invention include lactate, phosphate, citrate, acetate, tartrate and hydrochloric acid buffers. A preferred buffer is a lactate buffer. Non-limiting examples of the respective buffer components used in the present invention include lactic acid, phosphoric acid, citric acid, acetic acid, tartaric acid and hydrochloric acid. A preferred buffer component is a lactic acid.

Non-limiting examples of a bulking agent include HPbCD, dextran, sorbitol, glycine, mannitol, trehalose and sucrose. An alternative bulking agent is a combination of these excipients resulting in an amorphous structure of the cake. A preferred bulking agent is sucrose.

As is evident to those skilled in the art, the many of the deacetylase inhibitor compounds of the present invention contain asymmetric carbon atoms. It should be understood, therefore, that the individual stereoisomers are contemplated as being included within the scope of this invention.

The therapeutic compound(s) is present in the pharmaceutical compositions of the present invention in a therapeutically effective amount or concentration. Such a therapeutically effective amount or concentration is known to one of ordinary skill in the art as the amount or concentration varies with the therapeutic compound being used and the indication which is being addressed. For example, in accordance with the present invention, the therapeutic compound may be present in an amount by weight of up to about 20% by weight of the pharmaceutical composition, e.g., from about 0.01% by weight. The therapeutic compound may also be present in an amount from about 0.1-10% by weight of the pharmaceutical composition, from about 0.1% to about 5% by weight of the pharmaceutical composition.

A therapeutically effective amount of a therapeutic compound is mixed with an chelator/antioxidant, i.e., ETDA disodium; a buffer or buffer component, i.e., lactate buffer or lactic acid respectively; and a bulking agent, i.e., sucrose to form a solution. Calculated on a solution basis, the solution contains the therapeutic compound from 0.01-10% (w/v), e.g., 0.1-5% (w/v). Furthermore, the solution contains, e.g., a concentration of the chelator/antioxidant which is 0-2% (w/v), e.g., 0.01-0.1% (w/v). Furthermore, the solution contains a concentration of the buffer or buffer component from 0.01-10% (w/v), e.g. 0.05-0.5% (w/v). Furthermore, the solution contains, e.g., a concentration of the bulking agent from about 1% to about 50% (w/v), e.g., 5% to about 25%.

Once mixed, the solution is filled into a container that is suitable for lyophilization, e.g., a glass vial. The lyophilization cycle typically includes the following steps: a freezing step, a primary drying step and a secondary drying step.

In the freezing step, the solution is cooled. The temperature and duration of the freezing step is chosen such that all of the ingredients in the composition are completely frozen. For example, a suitable freezing temperature is approximately below −40° C. The water in the formulation becomes crystalline ice. The balance of the formulation in the frozen state may be crystalline, amorphous or a combination thereof.

In the primary drying step, the ice formed during freezing is removed by sublimation at sub-ambient temperatures (although greater than the freezing temperature) under vacuum. For example, the chamber pressure used for sublimation can be from about 40-400 milliTorr and the temperature be between −30° C. to −5° C. During the primary drying step, the formulation should be maintained in the solid state having product temperature below the collapse temperature (“T_{c}”) of the formulation. The T_{c} is the temperature above which the freeze-dried cake loses macroscopic structure and collapses during freeze-drying. For amorphous products the glass transition temperature (“T′_{g}”) or for crystalline products the eutectic temperature (“Tₑ”) are approximately the same as T_{c}. In addition, the T_{g} for the maximally freeze concentrated solution (“T′_{g}”) is important to the development of lyophilization cycles because this represents the highest temperature that is safe for the composition for primary drying.

After primary drying, any residual amounts of liquid which could not be removed by sublimation is removed by secondary drying, i.e., desorption. The temperature during secondary drying is near or greater than ambient temperature.

After lyophilization, the pharmaceutical composition becomes a cake. Such a cake should be pharmaceutically acceptable. As used herein, a “pharmaceutically acceptable cake” refers to a non-collapsed solid drug product remaining after lyophilization that has certain desirable characteristics, e.g., pharmaceutically acceptable, long-term stability, a short reconstitution time, an elegant appearance and maintenance of the characteristics of the original solution upon reconstitution. The pharmaceutically acceptable cake can be solid, powder or granular material. The pharmaceutically acceptable cake may also contain up to five percent water by weight of the cake.

It is understood that while the present invention has been described in conjunction with the detailed description thereof that the foregoing description is intended to illustrate and not limit the scope of the invention, which is defined by the scope of the following claims. Other aspects, advantages and modifications are within the scope of the claims.

### Example 1

### N-hydroxy-3-[4-[[[2-(2-methyl-1H-indol-3-yl)-ethyl]-amino]methyl]phenyl]-2E-2-propenamide, or a pharmaceutically acceptable salt thereof freeze dried formulation

| | |
|---|---|
| LBH589 lactate | 2.5 mg/mL |
| Sucrose | 5% |
| EDTA | 0.05% |
| Lactate buffer | 15 mM, pH 3.7 ± 0.2 |
| 2 mL filled in 6 mL vial | |

### Freeze Dried Cycle Using the Above-Identified Formulation

| Target temp | Soaking time | Ramp rate | Chamber pressure |
|---|---|---|---|
| (° C.) | (min) | (° C./min) | (micron) |
| −50 | 360 | 1 | |
| −40 | 1080 | 0.5 | 125 |
| −30 | 1080 | 0.5 | 125 |
| −20 | 1440 | 0.5 | 125 |
| 25 | 720 | 0.2 | 125 |

### Stability Profile for the Above-Identified Formulation

| | | | | Related substances RRT | | | | | | | | |
|---|---|---|---|---|---|---|---|---|---|---|---|---|
| | | | | 0.08 | | 0.67 | | 1.25 | | | Reconstitution | |
| | | | Assay LBH589 | OX1 | | OX2 | | Hyd | 1.74 | Total degs | time | Water |
| Batch | Condition | Timepoint | (90.0-110.0) | (≦0.84) | 0.09 | (≦0.87) | 1.22 | (≦1.4) | (≦0.5) | (≦4.2) | (seconds) | Content |
| TRD0966- | | Initial | 98.0% | | | | 0.05% | 0.47% | 0.05% | 0.56% | 15 | 1.99% |
| 121A2 | | | | | | | | | | | | |
| | 25/60 | 1 M | 97.5% | | | | 0.05% | 0.49% | 0.07% | 0.61% | 15 | 1.42% |
| | 40/75 | 1 M | 97.3% | | | | 0.05% | 0.52% | 0.16% | 0.73% | 38 | 2.10% |
| | 25/60 | 3 M | 95.6% | | | | 0.05% | 0.50% | 0.10% | 0.65% | 12 | 1.22% |
| | 40/75 | 3 M | 97.6% | ≦LOQ | | ≦LOQ | 0.05% | 0.60% | 0.28% | 0.93% | 13 | 1.60% |
| | 25/60 | 6 M | 96.8% | | | 0.05% | 0.05% | 0.51% | 0.12% | 0.73% | 15 | 1.43% |
| | 40/75 | 6 M | 97.2% | | 0.05% | 0.05% | | 0.69% | 0.43% | 1.22% | 15 | 2.39% |

### Example 2

### N-hydroxy-3-[4-[[[2-(2-methyl-1H-indol-3-yl)-ethyl]-amino]methyl]phenyl]-2E-2-propenamide, or a pharmaceutically acceptable salt thereof freeze dried formulation

| | |
|---|---|
| LBH589 | 3 mg/mL |
| Sucrose | 1% |
| Dextran | 4% |
| Lactate buffer | 50 mM |
| Phosphate buffer | 20 mM |

### Example 3

### N-Hydroxy-3-[4-[[[2-(2-methyl-1H-indol-3-yl)-ethyl]-amino]methyl]phenyl]-2E-2-propenamide or a pharmaceutically acceptable salt thereof freeze dried formulation

| | |
|---|---|
| LBH589 lactate | 2.5 mg/ml |
| HPbCD | 20% |
| Lactate buffer | 15 mM |

### Example 4

### Other Freeze Dried Cycles

| Target temp | Soaking time | Ramp rate | Chamber pressure |
|---|---|---|---|
| (° C.) | (min) | (° C./min) | (micron) |
| −50 | 360 | 1 | |
| −40 | 2160 | 0.1 | 50 |
| −30 | 1440 | 0.1 | 50 |
| 20 | 720 | 0.5 | 50 |

### Freeze Dried Cycles Using the Above-Identified Formulation

| Target temp | Soaking time | Ramp rate | Chamber pressure |
|---|---|---|---|
| (° C.) | (min) | (° C./min) | (micron) |
| 5 | 60 | 1 | |
| −5 | 30 | 1 | |
| −50 | 180 | 1 | |
| −15 | 1200 | 0.1 | 150 |
| 10 | 360 | 0.1 | 150 |
| 35 | 180 | 0.1 | 150 |
| 5 | 980 | 1 | 150 |

### Freeze Dried Cycles Using the Above-Identified Formulation

| Target temp | Soaking time | Ramp rate | Chamber pressure |
|---|---|---|---|
| (° C.) | (min) | (° C./min) | (micron) |
| 5 | 60 | | |
| −50 | 180 | 1 | |
| −31 | 3000 | 0.1 | 55 |
| 30 | 720 | 1 | 55 |

### Example 5

### N-hydroxy-3-[4-[[[2-(2-methyl-1H-indol-3-yl)-ethyl]-amino]methyl]phenyl]-2E-2-propenamide, or a pharmaceutically acceptable salt thereof freeze dried formulation

| | |
|---|---|
| LBH589 lactate | 1.887 mg/mL |
| Sucrose | 8.5% |
| Disodium Edetate dihydrate | 0.05% |
| Lactic acid DL | 1.351 mg/mL (15 mM) |
| 5 mL filled in 10 mL vial | |

### Freeze Dried Cycle Using the Above-Identified Formulation

| Target temp | Soaking time | Ramp rate | Chamber pressure |
|---|---|---|---|
| (° C.) | (min) | (° C./min) | (microbar) |
| −40 | 80 | 1 | |
| −40 | 180 | — | |
| −15 | 25 | 1 | 70 |
| −15 | 600 | — | 70 |
| −21 | 6 | 1 | 70 |
| −21 | 4560 | — | 70 |
| 30 | 51 | 1 | not set |
| 30 | 900 | — | not set |
| 20 | 10 | 1 | not set |
| 20 | 60 | — | not set |
| 20 | — | — | 800 mbar |

What is claimed is:

1. A pharmaceutical composition comprising:
N-hydroxy-3-[4-[[[2-(2-methyl-1H-indol-3-yl)-ethyl]-amino]methyl]phenyl]-2E-2-propenamide, or a pharmaceutically acceptable salt thereof;
a buffer or buffer component; and
a bulking agent.

2. The composition of claim 1, further comprising a chelator/antioxidant.

3. The composition of claim 1, wherein said bulking agent is selected from sucrose, trehalose, dextran and HPbCD.

4. The composition of claim 2, wherein said chelator/antioxidant is ETDA disodium.

5. The composition of claim 1, wherein said buffer or buffer component is selected from a lactate buffer or lactic acid, a phosphate buffer or phosphoric acid, and a combination of both.

6. The composition of claim 1 wherein said composition forms a pharmaceutically acceptable cake after lyophilization.

7. The composition of claim 1, wherein the pharmaceutically acceptable salt is a lactate salt.

8. A process of making a pharmaceutically acceptable cake comprising the steps of:
(a) forming a solution comprising N-hydroxy-3-[4-[[[2-(2-methyl-1H-indol-3-yl)-ethyl]-amino]methyl]phenyl]-2E-2-propenamide, or a pharmaceutically acceptable salt thereof, a chelator/antioxidant, a buffer and a bulking agent; and
(b) lyophilizing said solution to form a pharmaceutically acceptable cake.

9. The process of claim 8, wherein said bulking agent is selected from sucrose, trehalose, dextran and HPbCD.

10. The process of claim 9, wherein said bulking agent is sucrose.

11. The process of claim 8, wherein said chelator/antioxidant is ETDA disodium.

12. The process of claim 8, wherein said buffer or buffer component is selected from a lactate buffer (respectively lactic acid), a phosphate buffer (phosphoric acid respectively) and a combination of both.

13. The process of claim 12, wherein said buffer is a lactate buffer or wherein the said buffer component is lactic acid.

14. A pharmaceutically acceptable cake comprising:
(a) N-hydroxy-3-[4-[[[2-(2-methyl-1H-indol-3-yl)-ethyl]-amino]methyl]phenyl]-2E-2-propenamide, or a pharmaceutically acceptable salt thereof:
(b) a chelator/antioxidant, said anti-oxidant comprising from about 0% to about 5% by weight of the cake;
(c) a buffer or buffer component, said buffer or buffer component comprising from about 0.1% to about 15% by weight of the cake; and
(d) a bulking agent, said bulking agent comprising from about 50% to about 99.9% by weight of the cake.
